(19) United States
(12) Patent Application Publication
(10) Pub. No.: US 2010/0151293 A1
(43) Pub. Date: Jun. 17, 2010

(54) Method and apparatus for producing liquid hydrocarbons from coal

(76) Inventor: Andrew Hansen, Bullhead City, AZ (US)

Correspondence Address: JAMES E. BRUNTON, ESQ., P. O. BOX 29000, GLENDALE, CA, 91209, US

(21) Appl. No.: 12/590,550

(22) Filed: Nov. 10, 2009

Related U.S. Application Data
(60) Provisional application No. 61/201,837, filed on Dec. 15, 2008.

(51) Int. Cl. H01M 8/18; C10G 1/00; C07C 29/04; F02B 43/00

(52) U.S. Cl. 429/19; 208/425; 568/895; 60/39.12

(57) ABSTRACT

A method and apparatus for producing liquid hydrocarbons from coal that involves subjecting the coal to a pyrolysis reaction within a novel pyrolysis sub-system that includes a plurality of pyrolysis units, with each pyrolysis unit comprising two side-by-side retorts. To increase the amount of feedstock that can be handled within the required resident time, the plurality of pyrolysis units are uniquely arranged in tandem. The apparatus includes, in advance of the pyrolysis subsystem, an ash removal station wherein the ash is removed from the coal, a crushing station wherein the ash free coal is crushed to produce a crushed coal, and a desulfurizing steam station wherein the sulfur is substantially removed from the crushed coal. The apparatus further includes a closed fractionizing tower that is operably associated with the pyrolysis subsystem and into which the pyrolysis products from the pyrolysis subsystem pass. Within the closed fractionizing tower, the stream of pyrolysis condensate flowing from the pyrolysis subsystem is separated into various products that become the subject of further hydrogenation and treating. Additionally, the last pyrolysis unit of the pyrolysis subsystem is designed to drop the final residual material, or carbon char, from the pyrolysis subsystem into a quench and dry system and then into a water bath in a manner to maintain the oxygen-free environment. The collected char is then dried, bagged and forwarded to the fixed carbon market.

### CROSS-REFERENCE TO RELATED APPLICATIONS

This is a Non-Provisional Application claiming the benefit of co-pending Provisional Application No. 61/201,837 filed on Dec. 15, 2008.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates generally to a method and apparatus for processing coal. More particularly, the invention concerns a method and apparatus for producing liquid hydrocarbons from coal.

Coal, which is one of the most bountiful sources of fuel in the world, is typically found as a dark brown to black graphite-like material that is formed from fossilized plant matter. Coal generally comprises amorphous carbon combined with some organic and inorganic compounds. The quality and type of coal varies from high quality anthracite to bituminous to lignite which is softer than bituminous coal and comprises vegetable matter not as fully converted to carbon. Coal is burned in coal-fired plants throughout the world to produce energy in the form of electricity.

2. Description of Related Art Including Information Disclosed Under 37 CFR 1.97 and 1.98

The growing use of coal energy source, combined with concerns over climate change, is fueling the push for cleaner ways to produce energy from coal. Accordingly, many countries, including China and the United States are actively developing coal-to-oil technology. China is now engaged in the construction of a large coal-to-oil plant in Inner Mongolia, while the U.S. Department of Defense is looking at coal-to-oil technology as a means to reduce dependence on foreign oil. Senator Jim Bunning of Kentucky has introduced a bill calling for tax incentives for coal-to-liquid fuel conversion plants.

However, coal-to-oil plants as envisioned to date can be extremely expensive and raise serious environmental concerns about the prior art process's production of carbon dioxide, a greenhouse gas blamed for global warming, and other pollutants. These are among the drawbacks that the present invention seeks to overcome.

U.S. Pat. No. 6,013,158 issued to Wootten is exemplary of one type of prior art apparatus for forming liquid hydrocarbons from solid coal. The Wootten apparatus comprises means for pulverizing the coal to provide a particulate coal feed; means for extruding the coal feed to provide a hollow tube of compressed coal; means for extruding a clay feed to provide a hollow tube of compressed clay supported inside of the coal tube; means for burning a combustible fuel inside of the clay tube to fire and pyrolyze the extruded clay to produce hydrocarbon gases and coal char; and means for cooling the hydrocarbon gases to provide a liquid hydrocarbon product.

Another prior art patent dealing with the pyrolysis of coal is prior art U.S. Pat. No. 4,704,135 issued to Bonasso et al. This patent discloses an apparatus for converting coal to gas, liquid and solid products. In accordance with the Bonasso process the coal is subjected to a pyrolysis reaction at a temperature of at least about 260° C. in the presence of a hydrogen-containing gas, and the resultant solid residue is subjected to a gasification reaction with oxygen and steam at a temperature of at least about 482° C. thereby generating the necessary hydrogen-containing gas for the pyrolysis reaction and producing a solid product. Heat generated in the exothermic gasification reaction is transferred to the pyrolysis reaction, so the apparatus does not require any external source of heat except for means 88 to control the temperature of the gases passing to the pyrolysis reaction chamber. The gaseous fraction generated in the pyrolysis reaction is cooled to produce liquid and gas products, preferably after having first been subjected to a Fischer-Tropsch reaction.

Still another prior art method for recovering coal liquids is disclosed in U.S. Pat. No. 5,296,005 issued to Wolfe et al. The Wolfe et al. method comprises a method of recovering coal liquids and producing metallurgical coke that utilizes low ash, low sulfur coal as a parent for a coal char formed by pyrolysis with a volatile content of less than 8%. In accordance with the Wolfe et al. method, the char is briquetted and heated in an inert gas over a prescribed heat history to yield a high strength briquette with less than 2% volatile content.

### BRIEF SUMMARY OF THE INVENTION

It is an object of the present invention to provide a novel method and apparatus for producing liquid hydrocarbons from coal and like hydrocarbon materials. More particularly, it is an object of the invention to provide a method for producing liquid hydrocarbons from coal that involves subjecting the coal to a pyrolysis reaction within a novel a pyrolysis sub-system that includes plurality of pyrolysis units with each pyrolysis unit comprising two side-by-side retorts. To increase the amount of feedstock that can be handled within the required resident time, the plurality of pyrolysis units are uniquely arranged in tandem. Uniquely, the plurality of pyrolysis units are heated by an infra-red system that produces the required heat range of 950° to 1200° F. and concentrates the heat on the bottom one-third of each retort without impinging on the retort itself.

Another object of the invention is to provide an apparatus as described in the preceding paragraphs which includes, in advance of the pyrolysis subsystem, an ash removal station wherein the ash is removed from the coal, a crushing station wherein the ash free coal is crushed to produce a crushed coal, and a desulfurizing steam station wherein the sulfur is substantially removed from the crushed coal.

Another object of the invention is to provide an apparatus of the character described that includes a fuel cell sub-system that is operably associated with the pyrolysis subsystem and functions to produce electrical energy for use in operating the apparatus of the invention.

Another object of the invention is to provide an apparatus as described in the preceding paragraphs that further includes a closed fractionizing tower that is operably associated with the pyrolysis subsystem and into which the pyrolysis products from the pyrolysis subsystem pass. Within the closed fractionizing tower, the stream of pyrolysis condensate flowing from the pyrolysis subsystem is separated into various products that become the subject of further hydrogenation and treating.

Another object of the invention is to provide an apparatus as described in the preceding paragraphs that further includes a high temperature ceramic filter that is installed intermediate the closed fractionizing tower and the end of the last retort of the pyrolysis subsystem, the high temperature ceramic filter functioning to clean the gases received by the pyrolysis subsystem without lowering the temperature to a level that would initiate condensation.

Another object of the invention is to provide an apparatus of the class described that further includes a gas fired turbine generator that receives gases from the ceramic filter and functions to generate electricity that can be made available to the grid system of the apparatus.

Another object of the invention is to provide an apparatus of the character described in the preceding paragraphs in which the last pyrolysis unit of the pyrolysis subsystem is designed to drop the final residual material, or carbon char, from the pyrolysis subsystem into a quench and dry system and then into a water bath in a manner to maintain the oxygen-free environment. The collected char is then dried, bagged and forwarded to the fixed carbon market.

The forgoing, as well as other objects of the invention will be achieved by the method and apparatus of the invention as described in greater detail in the paragraphs that follow.

### BRIEF DESCRIPTION OF SEVERAL VIEWS OF THE DRAWINGS

FIGS. 1A and 1B when considered together comprise a generally diagrammatic view illustrating one form of the method and apparatus of the invention for producing liquid hydrocarbons from coal.

FIG. 2 is an enlarged side elevational view of the pyrolysis, or retort portion, of the apparatus of the invention shown in FIG. 1B.

FIG. 3 is an enlarged side elevational, diagrammatic view, partly broken away to show internal construction, of the feed module subassembly of the retort portion of the apparatus of the invention shown in FIG. 2.

FIG. 4 is an enlarged side elevational, diagrammatic view of one of the transition module subassemblies of the retort portion of the apparatus of the invention shown in FIG. 2.

FIG. 5 is an enlarged side elevational, diagrammatic view of the last, or gas take off module subassembly of the retort portion of the apparatus of the invention shown in FIG. 2.

FIG. 6 is a greatly enlarged cross-sectional, diagrammatic view taken along lines 6-6 of FIG. 3.

FIG. 7 is a greatly enlarged cross-sectional, diagrammatic view taken along lines 7-7 of FIG. 4.

### DETAILED DESCRIPTION OF THE INVENTION

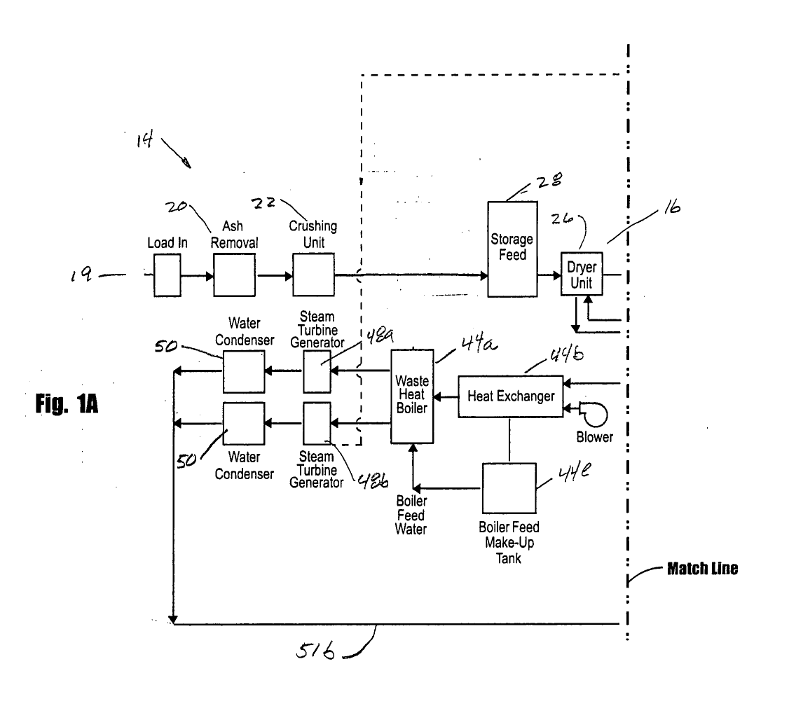

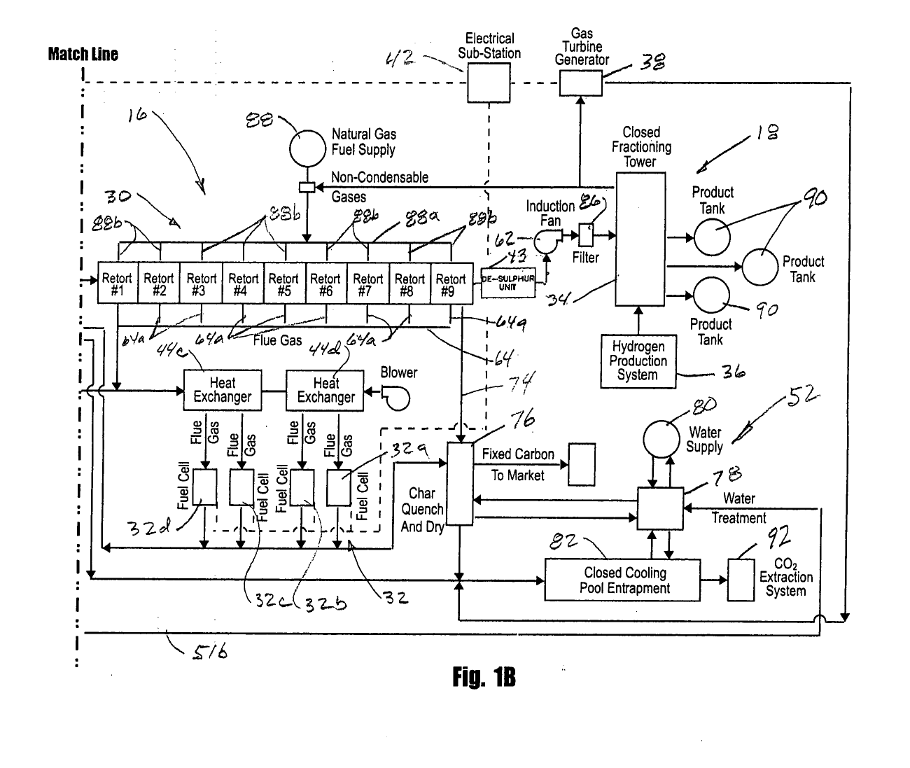

Referring to the drawings and particularly to FIGS. 1A and 1B, one form of the method and apparatus of the invention for producing liquid hydrocarbons from coal and like hydrocarbon materials is there shown in a generally diagrammatic form. The apparatus here comprises a material input section generally designated as 14, a pyrolysis, or retort section generally designated as 16 (FIG. 1B) and a fractionating section generally designated as 18.

The material input section 14 here includes a feed, or load-in station and an ash removal station 20 wherein the ash is removed from the coal in a conventional manner to produce a substantially ash free coal. Section 14 also includes a crushing station 22 wherein the ash free coal is crushed to produce a crushed coal.

The pyrolysis, or retort section 18 here includes a conventional dryer unit 26 which receives the desulfurized crushed coal from a storage unit 28 and functions to dry the desulfurized crushed coal. Section 18 also includes a plurality of pyrolysis units, or retorts 30, the details of construction of which will presently be described. The pyrolysis, or retort section 18 further includes a fuel cell system generally designated by the numeral 32 that is operably associated with the retorts 30 and functions to produce electrical energy in a conventional manner.

With regard to the pyrolysis section, pyrolysis may be defined as the chemical decomposition of organic materials by heating in the absence of oxygen or any other reagents. Pyrolysis long has been known to those skilled in the art of waste treatment as an effective process for reducing the organic components of a variety of compositions of waste material, such as conventional industrial and municipal waste, to products which present no harm to the atmosphere and which can be used in whole or in part to provide a useful source of energy or a material that can be recycled into a product having commercial value. One very successful pyrolytic waste treatment system is described in U.S. Pat. No. 5,868,085 in which the present inventor is named as a co-inventor.

The pyrolytic process employs high temperature in, most desirably, an atmosphere substantially free of oxygen (for example, in a practical vacuum) to convert the solid organic components of waste to other states of matter; such pyrosylates in a liquid or vapor phase.

A typical waste treatment system utilizing pyrolysis has an input structure for introducing the waste; a chamber or retort from which air can be purged and in which pyrolysis processing occurs; a feature for raising the temperature inside the chamber; an element that allows the vaporized waste material or “off-gases” to be vented to the environment, which element may or may not include some feature for cleaning or scrubbing the gases; and an assembly through which is discharged the solid or molten residue of the pyrolytic conversion process.

Other features often are provided to continuously move waste through the treatment unit while the system is being operated, such as a form of conveyance arrangement. Screw conveyors or conveyor belts oriented at an incline have been used to ramp waste material, in units of a defined volume and at a defined rate of flow, up from a storage bin or pre-treatment assembly at the ground level to a charging hopper at the top of the treatment unit through which waste is metered into the pyrolytic chamber. Screw conveyors, auger screws and worm conveyors all have been used to impel waste through the retort while pyrolysis takes place, again, to encourage predictable results from the process.

It is well known that the efficiency of pyrolysis is negatively affected by the presence of oxygen. One of the adverse effects oxygen has is to increase the degree to which the chemical reactions taking place during conversion are explosive, which explosiveness, in turn, increases the turbulence in the chamber and tends to result in the recombination of the released gases with the solid material being processed, making the conversion less complete and thus inefficient.

The manner in which the retort chamber is supplied with heat energy to sustain pyrolysis also can affect the efficiency with which the process can be carried out. For example, it has been found that uniform application of heat to the outer wall of the retort, through which it is conducted into the interior of the chamber, reduces the risk that the retort will buckle from uneven distribution of high temperatures and tends to encourage a more even distribution of heat and consistency of temperature throughout the chamber, which leads to consistent processing results. System features provided to address even heating have included those directed to the manner in which the primary source of heat energy, commonly fuel gases being combusted in a heating chamber, is arranged with relation to the retort, and the number and placement of fuel gas injection ports, etc.

Referring once again to FIG. 1B of the drawings, the fractionating section 18 of the invention here includes a conventional closed fractionating tower 34 and an operably associated hydrogen production system 36. Interconnected with the closed fractionating tower 34 is a conventional gas turbine generator 38. The turbine generator 38 and the fuel cells 32a, 32b, 32c and 32d of the fuel cell system 32 are operably interconnected with an electrical sub-station 42.

The process of one form of the invention involves the step of using the ash removal station 20 to first wash the coal that is delivered to the site as “mine run”, to remove ash, dirt, stones and like foreign matter. Next, using conventional hammer mills, jaw crushers, or like material reduction equipment, the washed product is crushed in the crushing unit 22 to produce a washed, crushed product having a size of about one half inch, or smaller.

As illustrated in FIG. 1B, the apparatus of the invention also includes a generating system that includes a waste heat boiler 44a, a series of heat exchangers 44b, 44c and 44d and a boiler feed make-up tank 44e (FIGS. 1A and 1B).

As illustrated in FIG. 1A of the drawings, the waste heat boiler 44a is interconnected with a pair of conventional steam turbine generators 48a and 48b, each of which is, in turn, connected to a second water condenser 50. Water condensers 46 and 50 are interconnected by conduits 51a and 51b with the water system 52 (FIG. 1B) the details of construction of which will presently be described.

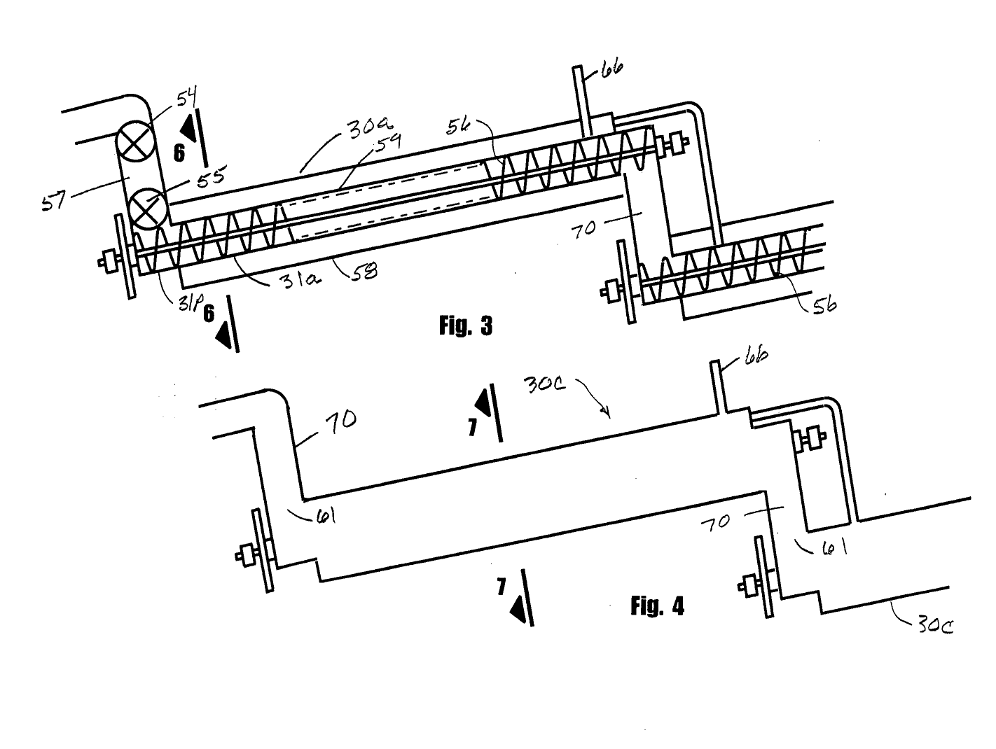

Following the crushing step, the material is transferred to the storage feed tank 28. From the storage feed tank the material is transferred to the previously identified dryer unit 26 and is stored in vibratory cone bottom tanks, each one of which will service one line of nine pyrolysis unit housings 30. Uniquely, the drying unit 26 is designed to receive waste heat from heat exchangers 44c and 44d. From the drying unit 26 weight feeders will then deposit precise quantities of the dried material onto a conveyer belt (not shown) that feeds the material into 2′×2′ openings in each air lock 54 of the first pyrolysis unit housing 30a (FIGS. 3 and 6) at a rate of about 0.4 cubic feet per second.

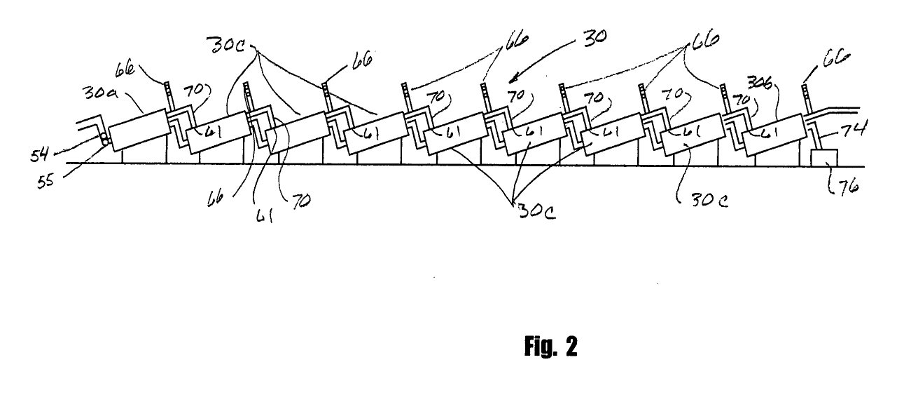

As illustrated in FIGS. 2, 6 and 7, the apparatus of the present form of the invention comprises a plurality of pyrolysis unit housings 30 arranged in tandem. In the present embodiment of the invention each pyrolysis unit housing 30 houses two side-by-side retorts 31a and 31b (FIG. 6) for a total of 18 retorts. However, depending on nature of the organics and the total cubic feet per day of material being processed, more or less retorts may be provided. In this regard, it should be noted that the initial prior art pyrolytic waste treatment systems were stationary “batch” reaction vessels. This approach was partially successful, but prone to explosive situations where off gases were not exhausted fast enough to maintain a negative pressure in the furnace.

The first “continuous flow” pyrolytic waste treatment systems made use of double chamber rotary valves at the entry and exit points of a centrally-located reaction vessel, termed a “retort.” To ensure completion of the pyrolytic reactions, the waste material was required to remain within the retort for a minimum specified time, termed the “required resident time.”

To increase the amount of feedstock that can be handled within the required resident time, designers tried increasing the length of the retort chamber which, when combined with an increase in the speed of the material through the chamber, would have permitted a greater amount of feedstock per unit of time. Unfortunately, experience revealed that if the chamber extends longer than about thirty feet, the material conveying screw sags, making contact with the walls of the retort chamber and causing the screw motor to fail.

For this reason, consideration was given towards expanding the diameter of the retort, again with the goal of increasing the amount of feedstock that could be pyrolyzed in a given amount of time. This design stratagem also proved a failure. Retort vessels of increasing diameter experienced a failure in the required particle movement resulting in the incomplete combustion of the organic particles.

This problem that plagued prior art pyrolytic waste treatment systems was uniquely solved by the present inventor by providing the novel tandem reactor design illustrated in FIG. 2 of the drawings.

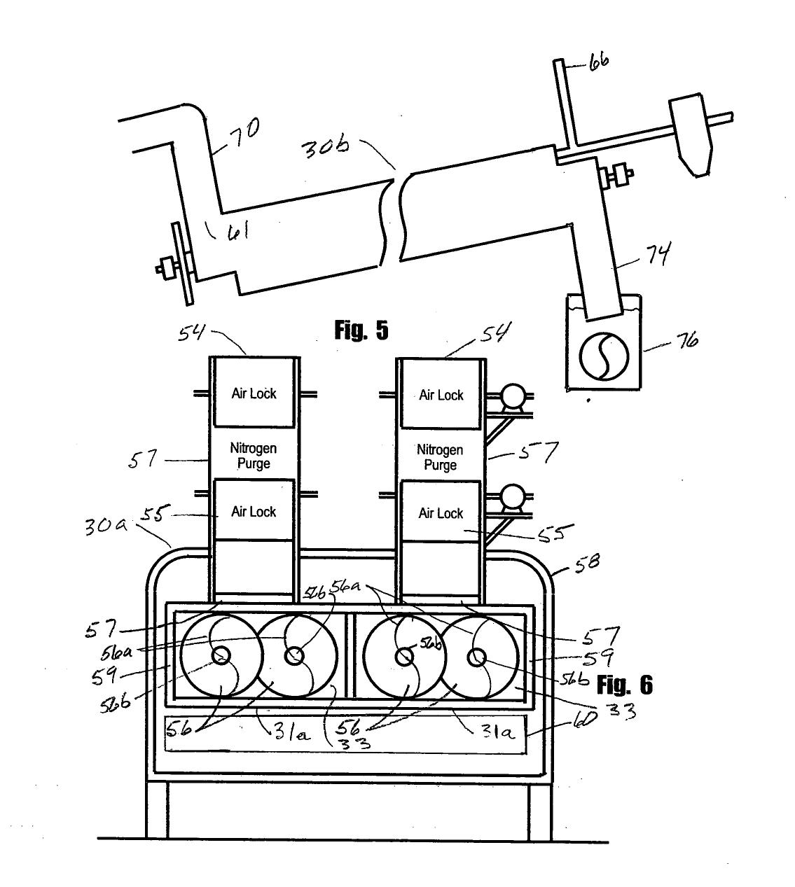

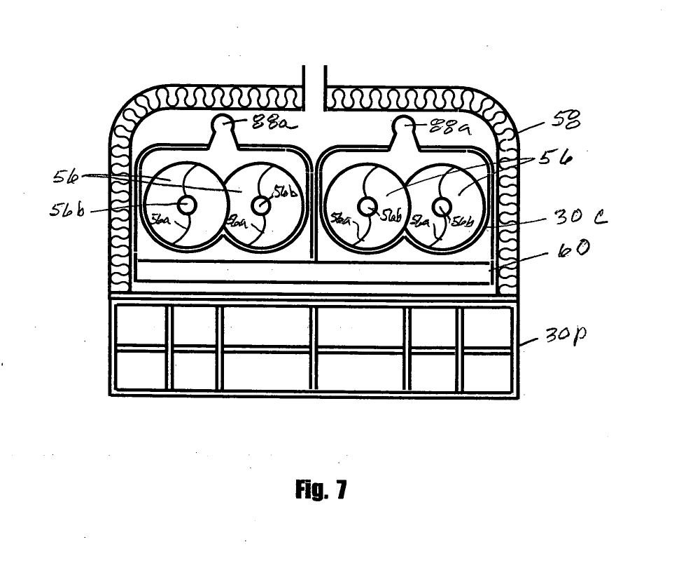

In the present form of the invention, each retort 31 of the plurality of tandem retorts (shown in the drawings as retorts 1-9) is about five feet wide and about 30 feet long and each retort includes two thirty inch diameter helical screws 56 (FIGS. 6 and 7), with one feed entry 57 to each retort (FIG. 6). The helical screws 56 are driven at a controlled rate of rotation by conventional motors (not shown) that are housed within each retort housing. All of the pyrolysis unit housings 30, which are supported by eight-inch I beam platforms 30p (FIG. 7), are of substantially the same configuration. However, pyrolysis unit housing 30a (FIGS. 3 and 6), and the last pyrolysis unit housing 30b, or retort #9 (FIGS. 1B and 5), are of the somewhat different construction shown in FIGS. 3 and 5, while intermediate pyrolysis unit housings 30c are of the general construction illustrated in FIGS. 4 and 7.

As best seen in FIGS. 3 and 6, a nitrogen infusion chute 57 having a length of approximately thirty-six inches is provided between the first and second rotary air locks 54 and 55. Rotary air locks 54 and 55 are of conventional construction, serve their normal function and are readily commercially available from various sources, including Sunco Power Systems Inc. of Charlotte, N.C.

With the construction described in the preceding paragraphs, nitrogen is infused into the chute 57 via a perforated pipe located immediately below the bottom of the first air lock 54 (not shown). Material introduced into the first rotary air lock 54 proceeds through the second rotary air lock 55 and drops by force of gravity into that portion 31p of the retorts 31a that extend past the insulated outer shell 58 (see, for example, FIG. 3).

Each retort 31 is housed within a stainless steel screw housing 59 (FIG. 6) and is heated by a conventional infra-red system 60 (FIGS. 6 and 7) that produces the required heat range of 950° to 1200° F. and concentrates the heat on the bottom one-third of the retort without impinging on the retort itself. Heat energy is provided/generated within the outer insulating housing 58, typically through the combustion of a petroleum product such as natural gas.

The various components that make up the conventional infra-red system 60 are readily commercially available from various sources, including Eclipse Power Equipment Co. of Richmond, Va. As previously mentioned, the operating temperature of each individual retort 31 can be raised or lowered as desired. An induction fan 62 located proximate the end of the last housing 30b ensures the maintenance of a negative pressure in the line of retorts and the evacuation of a minimum amount of particulate matter (FIG. 1B).

The material introduced into the air locks proceeds through the sequential pyrolytic converter and is decomposed by heat in an oxygen-free environment into various gas and solid components. The rotary air locks function to prevent the entry of oxygenated air into the pyrolytic chambers 33 of the first retorts 31a. Once introduced into the pyrolytic chambers of the retorts 31a via the rotary air locks, the helical conveyors 56 sequentially force the desulfurized material through the pyrolytic chambers 33 at a measured pace that is calculated to provide the appropriate resident time within the retort. As indicated in FIGS. 6 and 7, the helical conveyor 56 here comprises a blade 56a wrapped about a central shaft 56b.

In operation of the apparatus, the material passes via the transitional units which are of the construction illustrated in FIG. 4 from the first retort 31a to the second retort in line, and then sequentially to each of the retorts that make up the pyrolysis portion 16 via the inlets 61 provided on each retort (see FIG. 2).

Gases are carried continuously from each retort through a gas plenum 64 that is attached to the screw housing 59 by a series of transfer pipes 64a (FIG. 1B). In this regard, it is to be noted that all metals used in the retort, gas plenum, retort shell and screws are of the same A.S.T.M. number to maintain the same co-efficient of expansion throughout the system.

Ports 66 are installed proximate the ends of the pyrolysis unit housings, both to sample the gas make-up or to remove the gas at that point if desired. For example, if mercury is discovered as a contaminant it can be removed and condensed as a solid/liquid by distillation-condensation at a certain point as it moves through the multiple retorts.

In the preferred form of the invention, the total retention time of the material within the retorts is approximately 27 minutes. This means that the material will only remain in each retort for approximately 3 minutes after which it then drops through the drop-out chute 70 that extends past the insulated outer shell on the end of the retort and into the receiving end or input 61 of the following retort.

The last pyrolysis unit housing 30b is designed to drop the final residual material which comprises a carbon char, through a drop-out chute 74 into a quench and dry system 76 (FIG. 5). From the quench and dry system 76 the char is transferred to a water bath 78 in a manner to maintain the oxygen-free environment. As seen in FIG. 1B, water bath 78 forms a part of the water system 52 which also includes a water supply 80 and a closed cooling pool entrapment 82. After drying, the char is dried and bagged and forwarded to the fixed carbon market. Quench and dry system 70 is of conventional construction and is readily commercially available from various sources, including the Lundell Manufacturing Company of Cherokee, Iowa.

From retort 31b, or retort #9, the material is treated to remove harmful sulfur. In this regard, over the years it has been recognized that sulfur, which is a particularly troublesome impurity in coal, can be from trace amounts up to about 7 percent by weight. Sulfur may be found in coal in various forms, e.g., organic sulfur, pyritic sulfur, or sulfate sulfur. In accordance with one form of the method of the present invention, sulfur is removed from the pyrolyzed product in the filter type desulfurizing station 43 (FIG. 1B). Desulfurizing station 43 is readily commercially available from the Pall Shumacher Company of Crailshaim, Germany. This company should be consulted for the details of construction and operation of the filter-type desulfurizing station 34.

A high temperature ceramic filter 86 is installed proximate the end of the last retort to clean the gases and to remove fine carbon from the gas stream and to remove any residual sulfur there from without lowering the temperature to a level that would initiate condensation. Ceramic filter 86 is of conventional construction and is readily commercially available from various sources, including Caldo Engineering of Bromsgrove, England.

Gases flow from the ceramic filter 86, to the closed fractionating tower 34, and to the previously mentioned gas fired turbine generator 38, to generate electricity that can be made available to the grid system.

After filtration, the desulfurized pyrolysis products pass to the closed fractionizing tower 34 wherein the stream of pyrolysis condensate is separated into various products that become the subject of further hydrogenation and treating. Fractioning tower 34 is of conventional construction and operation and is readily commercially available from various sources, including the Service and Technology Corp. of Bartlesville, Okla. In processing the pyrolysis products, the overhead distillate from the tower 34 is cooled and the distillate is typically separated into pyrolysis gas, tar water and light hydrocarbon condensate. Normally, part of the condensate is returned as reflux to the top of the tower and the excess is taken off as product.

In processing the pyrolysis gas, the composition and quality of the paralysis gas changes quite sharply. The long-term action of high temperatures on the absorption oil and the continuous increase in content of heavy hydrocarbons extracted from the paralysis products into the oil will bring about partial polymerization of the material and hence an increase an increase in viscosity.

As depicted in FIG. 1B, the non-condensable gases such as hydrogen and carbon monoxide are removed at the top of the fractioning tower 34 for use in ethanol production. A portion of the hydrogen can also be used to upgrade yield of liquid petroleum products in the fractioning tower and to blend with natural gas from the natural gas supply 88 for use in heating the retorts 31 via a gas plenum 88a that includes connector pipes 88b (FIG. 1B). The liquid petroleum products in the fractioning tower that are derived from the pyrolysis products are transferred to a series of product tanks 90 for periodic removal and appropriate disposition.

During operation, the apparatus of the invention, the previously mentioned electrical sub-station 42, will acquire all the electrical energy derived from the fuel cells, the gas turbine generator and the steam generator, and make any surplus available to the grid system.

As illustrated in FIGS. 1A and 1B, a portion of the flue gases will be fed to the dryer unit 26 to dry the feed material. The balance will be used to supply heat energy to the series of fuel cells 32a, 32b, 32c and 32d via heat exchangers 44c and 44d. Any residual heat will be used to heat the waste heat boiler 44a that will supply steam to power the steam turbine generator 48a. The fuel cells 30 are of conventional construction and are readily commercially available from various sources, including United Technologies Corporation of Hartford, Conn. Similarly, heat exchangers 44c and 44d are of conventional construction and are readily commercially available from various sources, including Hayden Industrial Products of Corona, Calif.

Any waste residual gases will be diverted to a covered pool entrapment 82 and a carbon dioxide extraction system 92 will be appropriately associated with the covered pool entrapment 82, to remove the carbon dioxide gas and containerize it for shipment.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

1. An apparatus for producing liquid hydrocarbons from a hydrocarbon material comprising:
(a) a material input section including:
(i) an ash removal station for removing ash from the hydrocarbon material to produce a substantially ash free hydrocarbon material; and
(ii) a crushing station for crushing the ash free hydrocarbon material to produce a crushed hydrocarbon material; and
(b) a pyrolysis section including:
(i) a dryer unit for drying said crushed hydrocarbon material to produce a substantially dry crushed hydrocarbon material;
(ii) a plurality of inclined pyrolysis unit housings arranged in tandem for pyrolizing said substantially dry crushed hydrocarbon material to produce a pyrolyzed product; and
(iii) a fuel cell system operably associated with said plurality of pyrolysis unit housings for producing electrical energy; and
(c) a fractionating section comprising a closed fractioning tower and a hydrogen production system operably associated with said closed fractionating tower for producing hydrogen.

2. The apparatus as defined in claim 1 in which said material input section further includes a storage unit connected to said crushing station for storing said crushed hydrocarbon material.

3. The apparatus as defined in claim 1 in which said pyrolysis section further includes a gas fired infra-red system operably associated with said plurality of pyrolysis unit housings for heating said plurality of pyrolysis unit housings.

4. The apparatus as defined in claim 1 in which said pyrolysis section further includes a filter type desulfurizing station connected to said plurality of inclined paralysis unit housings for removing sulfur from said pyrolyzed product to produce a desulfurized product.

5. The apparatus as defined in claim 1 in which said pyrolysis section further includes a high temperature ceramic filter operably interconnected connected with said filter type desulfurizing station for removing any residual sulfur from said desulfurized product.

6. The apparatus as defined in claim 1 in which each said pyrolysis unit housing houses two side by side retorts, each retort including two helical screws.

7. The apparatus as defined in claim 1 further including a steam generating system operably associated with said pyrolysis unit housings, said steam generating system including a waste heat boiler, a pair of steam turbine generators interconnected with said waste heat boiler and a series of heat exchangers operably associated with said waste heat boiler.

8. The apparatus as defined in claim 1 further including a steam generating system operably associated with said pyrolysis unit housings, said steam generating system including a waste heat boiler, a pair of steam turbine generators interconnected with said waste heat boiler and a water condenser connected to each of said steam turbine generators.

9. The apparatus as defined in claim 1 in which said fractionating section further includes a gas turbine generator interconnected with said closed fractionating tower.

10. An apparatus for producing liquid hydrocarbons from a hydrocarbon material comprising:
(a) a material input section including:
(i) an ash removal station for removing ash from the hydrocarbon material to produce a substantially ash free hydrocarbon material;
(ii) a crushing station for crushing the ash free hydrocarbon material to produce a crushed hydrocarbon material; and
(iii) a storage unit connected to said crushing station for storing said crushed hydrocarbon material;
(b) a pyrolysis section including:
(i) a dryer unit for drying said crushed hydrocarbon material to produce a substantially dry crushed hydrocarbon material;
(ii) a plurality of inclined pyrolysis unit housings arranged in tandem for pyrolizing said substantially dry crushed hydrocarbon material to produce a pyrolyzed product, each said pyrolysis unit housing comprising two side by side retorts, each retort including two helical screws;
(iii) a gas fired infra-red system operably associated with said plurality of inclined pyrolysis unit housings for heating said plurality of pyrolysis unit housings;
(iv) a filter type desulfurizing station connected to said plurality of inclined pyrolysis unit housings for removing sulfur from said pyrolyzed product to produce a desulfurized product; and
(v) a fuel cell system operably associated with said plurality of pyrolysis unit housings for producing electrical energy; and
(c) a fractionating section comprising a closed fractioning tower and a hydrogen production system operably associated with said closed fractionating tower for producing hydrogen.

11. The apparatus as defined in claim 10 in which said pyrolysis section further includes a high temperature ceramic filter operably interconnected connected with said filter type desulfurizing station for removing any residual sulfur from said desulfurized product.

12. The apparatus as defined in claim 10 further including a steam generating system operably associated with said pyrolysis unit housings, said steam generating system including a waste heat boiler, a pair of steam turbine generators interconnected with said waste heat boiler and a series of heat exchangers operably associated with said waste heat boiler.

13. The apparatus as defined in claim 10 further including a steam generating system operably associated with said pyrolysis unit housings, said steam generating system including a waste heat boiler, a pair of steam turbine generators interconnected with said waste heat boiler and a water condenser connected to each of said steam turbine generators.

14. The apparatus as defined in claim 10 in which said fractionating section further includes a gas turbine generator interconnected with said closed fractionating tower.

15. A method for producing liquid hydrocarbons from hydrocarbon material using an apparatus that includes an ash removal station, a crushing station, a dryer unit, a plurality of inclined pyrolysis unit housings arranged in tandem, a filter type desulfurizing station and a fractionizing tower, the method comprising the steps of:
(a) using the ash removal station, removing ash from a hydrocarbon material to produce a substantially ash free hydrocarbon material;
(b) using the crushing station, crushing the substantially ash free hydrocarbon material to produce a crushed material;
(c) using the drying unit, drying the crushed material to produce a substantially dry crushed material;
(d) using the plurality of inclined pyrolysis unit housings, paralyzing a substantially dry crushed material to produce a pyrolized product;
(e) using the a filter type desulfurizing station removing sulfur from the pyrolized product to produce a desulfurized pyrolized product including a pyrolysis condensate; and
(f) using the fractionizing tower separating the desulfurized pyrolized product and the pyrolysis condensate into various products, including liquid hydrocarbons.

16. The method as defined in claim 15 including the further step of using a high temperature ceramic filter removing any residual sulfur from the desulfurized pyrolized product.

17. The method as defined in claim 15 including the further step of using a portion of the desulfurized pyrolized product to produce ethanol.

18. The method as defined in claim 15 including the further step of using a portion of the desulfurized pyrolized product to heat the pyrolysis unit housings.

19. The method as defined in claim 15 including the further step of using a portion of the desulfurized pyrolized product to drive a gas turbine generator that is interconnected with the closed fractionating tower.

20. The method as defined in claim 15 including the further step of using a portion of the desulfurized pyrolized product to produce electricity.
